(19) United States
(12) Patent Application Publication
(10) Pub. No.: US 2006/0286050 A1
(43) Pub. Date: Dec. 21, 2006

(54) Moisturizing lipstick compositions

(75) Inventors: Wei Hong Yu, Edison, NJ (US); Shirley Scherer, Nutley, NJ (US)

Correspondence Address: L'OREAL USA/ PATENT DEPARTMENT, 30 TERMINAL AVENUE, CLARK, NJ, 07066, US

(73) Assignee: L'OREAL, Paris (FR)

(21) Appl. No.: 11/443,860

(22) Filed: May 31, 2006

Related U.S. Application Data
(60) Provisional application No. 60/686,473, filed on Jun. 1, 2005.

(51) Int. Cl. A61K 8/64

(52) U.S. Cl. 424/63; 424/70.14

(57) ABSTRACT

A moisturizing cosmetic composition containing at least one oil gelling agent; at least one wax component; at least one polar oil; at least one active ingredient; and optionally, at least one colorant; and wherein the at least one wax component and at least one polar oil are present in a ratio by weight of from about 1:1 to about 1:5. The invention further relates to a process of moisturizing keratinous tissue comprising contacting the keratinous tissue with the moisturizing cosmetic composition.

### CROSS REFERENCE TO RELATED APPLICATION

This application claims the priority benefit of U.S. Provisional Patent Application No. 60/686,473, filed Jun. 1, 2005, the entire content of which is hereby incorporated by reference.

### BACKGROUND OF THE INVENTION

To protect the lips against drying out, lip balms in stick form are generally used. These balms contain treating oils, generally of plant origin such as triglycerides (see in this respect document JP-A-04,360,810). Unfortunately, even in large amounts, these oils do not treat the lips sufficiently, especially when they are swollen, cracked or chapped. Moreover, the use of large amounts of these oils makes the balm greasy. This in turn gives the lips an unpleasant aesthetic appearance and causes the balm to feel greasy or heavy upon application thereby yielding mediocre cosmetic properties.

Yet another disadvantage associated with the use of the above-described oils relates to the presence of solid actives within the stick such as, for example, UV filters. The addition of solid active protecting agents in these softer sticks causes the solid active to migrate to the bottom of the composition thereby inhibiting its performance. Moreover, the addition of harder waxes to improve the structure of the stick causes the stick to be too hard resulting in a negative impact in pay off. It is thus an object of the present invention to provide a moisturizing cosmetic composition for use on lips, in solid form, which does not suffer from the above-disclosed drawbacks.

### BRIEF SUMMARY OF THE INVENTION

The present invention relates to a moisturizing cosmetic composition containing:
(a) at least one oil gelling agent;
(b) at least one wax component;
(c) at least one polar oil;
(d) at least one active ingredient; and
(e) optionally, at least one colorant,

wherein the at least one wax component and the at least one polar oil are present in the composition in a ratio by weight of from about 1:1 to about 1:5.

The invention also relates to a process for moisturizing a keratinous tissue involving contacting the tissue with a composition containing:
(a) at least one oil gelling agent;
(b) at least one wax component;
(c) at least one polar oil;
(d) at least one active ingredient; and
(e) optionally, at least one colorant,

wherein the at least one wax component and the at least one polar oil are present in the composition in a ratio by weight of from about 1:1 to about 1:5.

### DETAILED DESCRIPTION

Other than in the operating examples, or where otherwise indicated, all numbers expressing quantities of ingredients and/or reaction conditions are to be understood as being modified in all instances by the term “about”.

It has been surprisingly discovered that the combination of at least one gelling agent, at least one wax component, at least one polar oil, at least one active ingredient and, optionally, at least one colorant, in a ratio by weight of wax component to polar oil of from about 1:1 to about 1:5, results in the formation of a moisturizing composition, preferably in stick form, which delivers excellent hydration onto keratinous tissue while maintaining its integrity. Moreover, in the event that an active ingredient is employed in solid form, the present invention will inhibit the settling of the active ingredient within the composition.

Without intending to be bound by theory, it is believed that by employing a certain ratio of wax component to polar oils, enhanced moisturization of keratinous tissue can be obtained due to the polar oils affinity for water.

The invention may be used as an eyeshadow, an eyeliner, a blush, or a lip composition. In addition, the product of the invention, instead of being molded in the form of a stick, may also be hot-poured.

By the term “keratinous tissue” it is meant the protective keratin containing layers of a body which includes, but is not limited to, skin and lips.

Moreover, by “comprising” it is meant that other steps and/or ingredients which do not affect the end result may be added. The products, compositions, methods and processes of the present invention can include all the essential elements and limitations of the invention described herein as well as any of the additional or optional ingredients, components, steps, or limitations described herein.

All percentages, parts and ratios are based upon the total weight of the compositions of the present invention unless otherwise indicated.

The compositions and processes, including the essential and additional or optional components are described in detail below.

Oil Gelling Agent

Suitable oil gelling agents include, but are not limited to, organomodified clays, particularly those clays treated with compounds chosen from quaternary amines and tertiary amines. Representative organomodified clays include organomodified bentonites, such as those sold under the name “Bentone 34” by the company Rheox, or organomodified hectorites, such as those sold under the name “Bentone 27” or “Bentone 38” by the company Rheox. In the event that the organomodified clay is employed in powder form, an activating solvent is required in order to form a gel. Suitable activating solvents include, but are not limited to, propylene carbonate, water and alcohol. The gel structure thus formed may be modified by varying the ratio of organomodified clay to activating solvent. This ratio may range from about 15:1 to about 5:1 w/w. It will result in a viscosity which may range from about 15 Pa.s to about 80 Pa.s, preferably from about 20 to about 70 Pa.s, as measured on a Rheomat viscometer, at 25° C., using spindle #3 at a shear rate of 200/sec for 10 minutes.

Other oil gelling agents include, but are not limited to, fat soluble alkylated guar gums such as hydroxypropyl guar gum, or cellulose gums such as methyl hydroxyethyl cellulose, cetyl hydroxyethyl cellulose, hydroxypropyl cellulose, or hydroxyethyl cellulose, and treated hydrophobic silicas. Furthermore, fatty acid salts such as aluminum, calcium or magnesium stearates, and N-acyl amino acids can also be used.

The oil gelling agent will typically be employed in an amount of from greater than about 0% to about 10% by weight, preferably from about 0.1% to about 5% and more preferably from about 0.5% to about 1.5% by weight, based on the weight of the composition.

Wax Component

Suitable wax components for use in the present invention include, but are not limited to, waxes of animal, vegetable and synthetic origin. These waxes typically possess a melting point of between about 40° C. and about 110° C., inclusive, and have a needle penetration at 25° C. of between 3 and 40, inclusive, as measured according to French standard NF T 60-123 or U.S. standard ASTM D 1321, at a temperature of 25° C. According to these standards, needle penetration is the measurement of the depth, expressed in tenths of a millimeter, to which a standardized needle, weighing 2.5 g, placed in a mobile assembly weighing 97.5 g and placed on the wax to be tested for 5 seconds, penetrates into the wax.

Representative waxes of animal origin include, but are not limited to, beeswaxes, lanolin waxes and Chinese insect waxes.

Waxes of vegetable origin include, but are not limited to, rice waxes, carnauba, candelilla or ouricury waxes, cork fiber waxes, sugar cane waxes, japan waxes, sumach wax and cottonseed wax.

Representative waxes of mineral origin include, but are not limited to, paraffin waxes, microcrystalline waxes, montan waxes and ozokerites.

Suitable waxes of synthetic origin include, but are not limited to, polyolefin waxes and in particular polyethylene waxes, those waxes obtained by Fischer-Tropsch synthesis, waxy copolymers and esters thereof, and silicone waxes.

It is also possible to use hydrogenated oils of animal or vegetable origin which still correspond to the physical characteristics mentioned above.

Representative hydrogenated oils include, but are not limited to, hydrogenated jojoba oils and hydrogenated oils which are obtained by catalytic hydrogenation of fatty substances comprising a chain chosen from linear and nonlinear C₈-C₃₂ fatty chains, hydrogenated sunflower oil, hydrogenated castor oil, hydrogenated coconut oil, hydrogenated lanolin and hydrogenated palm oils.

The waxes which can be used according to the present invention are preferably solid and stiff at a temperature of less than about 50° C.

The wax component will typically be employed in an amount of from greater than about 0% to about 30% by weight, preferably from about 1% to about 20% by weight, and more preferably from about 1% to about 15% by weight, based on the weight of the composition.

Polar Oil

The polar oils which may be used in the present invention include, but are not limited to:

hydrocarbon-based oils of plant origin, such as liquid triglycerides of fatty acids having 4 to 10 carbon atoms, such as triglycerides of heptanoic or octanoic acids, or alternatively sunflower oil, grapeseed oil, sesame oil, corn oil, apricot oil, castor oil, avocado oil, olive oil, cereal germ oil, soybean oil, sweet almond oil, palm oil, rapeseed oil, cotton oil, hazelnut oil, macadamia oil, jojoba oil, caprylic/capric acid triglycerides, such as those sold under the names Miglyol 810, 812 and 818 by the company Dynamit Nobel, and karite or shea butter;

synthetic esters and ethers, such as the oils of formula R₁COOR₂ in which R₁ represents a higher fatty acid residue containing from 6 to 29 carbon atoms and R₂ represents a hydrocarbon-based chain containing from 3 to 30 carbon atoms, such as Purcellin oil, isopropyl myristate, isopropyl palmitate, butyl stearate, hexyl laurate, diisopropyl adipate, isononyl isononanoate, 2-ethylhexyl palmitate, 2-hexyldecyl laurate, 2-octyldecyl palmitate, and 2-octyldodecyl myristate or lactate; esters such as bis-diglyceryl polyacyladipate-2; polyol esters, such as propylene glycol dioctanoate, neopentyl glycol heptanoate, diethylene glycol diisononanoate, polyglyceryl di-isostearate, pentaerythritol esters; dimer acid esters such as diisopropyl dimer dilinoleate and diisostearyl dimer dilinoleate commercially available from Noveon as Schercemol DID and Schercemol DISD, respectively;

fatty alcohols which are liquid at room temperature, with a branched and/or unsaturated carbon chain containing from 12 to 26 carbon atoms, such as octyldodecanol, isostearyl alcohol, oleyl alcohol, 2-hexyldecanol, 2-butyloctanol and 2-undecylpentadecanol;

sterols and derivatives such as cholesterol, PEG-5 Rapeseed sterol, PEG-10 Rapeseed sterol, PEG derivatives of phytosterols or soy sterols;

fatty acid blends such as those sold under the denomination of Prolipid sold by ISP, which are available as Prolipid 131, 141, 151, and 161, and which are a mixture of Glyceryl Stearate SE and fatty acids such as palmitic and stearic acid, and/or fatty alcohols, such as behenyl alcohol and cetearyl alcohol, and/or maleated soybean oil and/or C₁₂-C₁₆ alcohols and/or lecithin.

The polar oils can be present in the composition according to the invention in an amount ranging from about 0.1% to about 65% by weight, such as from about 10% to about 45% by weight, and such as from about 20% to about 40% by weight, based on the weight of the composition.

The compositions of the present invention will typically contain the at least one wax component and the at least one polar oil in a ratio by weight of from about 1:1 to about 1:5, preferably from about 1:2 to about 1:5, preferably from about 1:2 to about 1:4, and more preferably from about 1:3 to about 1:4.

Active Ingredients

Examples of suitable active ingredients which may be used include, but are not limited to, vitamin C, alpha hydroxy acids, calamine, allantoin, refreshing active agents such as menthol, proteins and protein hydrolysates, sugar and derivatives thereof, glycyrrhetinic acid, and UV blockers. According to a preferred embodiment of the present invention, the active ingredient is employed in solid form. A particularly preferred active ingredient, in solid form, is allantoin.

The active ingredient will typically be present in the composition in an amount of from greater than 0% to about 10% by weight, preferably from about 0.1% to about 5% by weight, and more preferably from about 0.1% to about 1% by weight, based on the weight of the composition.

Optional Ingredients

The composition can also comprise at least one colorant, such as pulverulent compounds and/or fat-soluble dyes. The pulverulent compounds can be chosen from pigments, pearlescent agents and fillers commonly used in cosmetic or dermatological compositions.

The pigments can be chosen from white and colored, inorganic and organic pigments. Representative inorganic pigments include titanium dioxide, which is optionally surface treated, or zirconium or cerium oxides, as well as iron or chromium oxides, manganese violet, ultramarine blue, chromium hydrate and ferric blue. Representative organic pigments include carbon black, pigments of D & C type, and lakes based on cochineal carmine or on barium, strontium, calcium or aluminum.

The pearlescent pigments can be chosen from white pearlescent pigments, such as mica covered with titanium oxide or with bismuth oxychloride, colored pearlescent pigments, such as titanium dioxide-coated mica with iron oxides, titanium dioxide-coated mica with in particular ferric blue or chromium oxide, or titanium dioxide-coated mica with an organic pigment of the abovementioned type, and pearlescent pigments based on bismuth oxychloride.

The fillers can be chosen from those well-known to a person skilled in the art as commonly used in cosmetic compositions.

The colorant will typically be present in the composition in an amount of from about 0.1% to about 65% by weight, preferably from about 0.1% to about 40% by weight, and more preferably from about 0.1% to about 20% by weight, based on the weight of the composition.

Other oils which may be used in conjunction with the polar oils include hydrocarbon-based oils of animal origin, such as perhydrosqualene; linear or branched hydrocarbons of mineral or synthetic origin, such as petroleum jelly, polydecenes and hydrogenated polyisobutenes, such as Parleam; and mixtures thereof.

These other oils can be present in the composition according to the invention in an amount ranging from about 0.1% to about 65% by weight, preferably from about 0.1% to about 40% by weight, and more preferably from about 0.1 to about 20% by weight, based on the weight of the composition.

Humectants which may be used include, but are not limited to, niacinamide, PCA soda, sodium lactate, amino acids, urea, panthenol, sodium hyaluronate, collagen, chondroitin sulfate, elastin, pectin, carrageenan, trehalose, sodium alginate, chitin and derivatives, chitosan and derivatives.

The moisturizing composition may also contain oil soluble film formers, i.e. ones that are compatible with the oil/wax phase and form a film after application. Representative examples include, but are not limited to, VP/hexadecene copolymer, PVP/hexadecene copolymer, VP/Eicosene copolymer, trimethylsiloxysilicate, acrylates copolymer.

The moisturizing composition may comprise sunscreen agents which work either as physical blockers or as chemical absorbers. Physical blockers reflect or scatter ultraviolet radiation. Typical examples of physical blockers include, but are not limited to, red petrolatum, titanium dioxide, and zinc oxide. Chemical absorbers are classified, depending on the type of radiation they protect against, as either UV-A or UV-B absorbers. UV-A absorbers generally absorb radiation in the 320 to 400 nm region of the ultraviolet spectrum. UV-A absorbers include, but are not limited to, anthranilates, benzophenones, and dibenzoyl methanes. UV-B absorbers generally absorb radiation in the 280 to 320 nm region of the ultraviolet spectrum. UV-B absorbers include p-aminobenzoic acid derivatives, camphor derivatives, cinnamates, and salicylates.

According to yet another embodiment of the present invention, there is also provided a process for moisturizing keratinous tissue in an aesthetically pleasing manner involving contacting the keratinous tissue with the above-disclosed composition. By including at least one colorant in the composition of the invention, a moisturizing lipstick can be formulated which, when applied onto the lips, moisturizes them in an aesthetically pleasing manner.

The composition can additionally comprise any additive generally used in such compositions, such as preservatives, fragrances, agents for combating free radicals, moisturizing agents, vitamins, proteins, ceramides or plasticizers.

The hardness of the finished composition is measured using the “cheese wire” method, which involves cutting an 8.1 mm tube of composition and measuring its hardness at 20° C. using a DFGHS 2 tensile testing machine from Indelco-Chatillon Co. at a speed of 10 mm/minute. The hardness value from this method is expressed in grams as the shear force required to cut a stick under the above conditions. According to this method, the hardness of compositions according to the present invention may, for example, range from about 100 g to about 200 g, preferably from about 115 g to about 155 g, and more preferably from about 130 g to about 140 g.

The hardness of the composition of the present invention may be such that the compositions are self-supporting and can easily leave a satisfactory deposit on a keratinous material. In addition, this hardness may impart good impact strength to the inventive compositions which may be molded or cast, for example, in stick or dish form.

The skilled artisan may choose to evaluate a composition using the test for hardness outlined above based on the application envisaged and the hardness desired. If one obtains an acceptable hardness value, in view of the intended application the composition falls within the scope of the invention.

The present invention is further described in terms of the following non-limiting examples. Unless otherwise indicated, all parts and percentages are on a weight-by-weight percentage basis.

### EXAMPLE 1

A lipstick in accordance with the present invention was made by preparing an oil phase, a gel phase and an additive phase which were mixed together to make a preparatory phase. The finished product was then made by mixing the preparatory phase with the fragrance.

Oil Phase:

Equipment: Mixing kettle

Ingredients and preparation procedure

The following ingredients were combined into the mixing kettle and heated to 85-90° C. under medium agitation: Combine the following ingredients into the mixing kettle: 18.31 g Polyglyceryl-2-Diisostearate, 6.86 g Diisopropyl Dimer Dilinoleate, 27.12 g Hydrogenated Polyisobutene, 11.44 g Bis-Diglyceryl Polyacyladipate-2, 10.76 g Squalane, 10.76 g Simmondsia Chinensis (Jojoba) Seed Oil, 6.86 g Butyrospermum Parkii (Shea Butter), 4.58 g VP/Hexadecene Copolymer, 0.92 g Parabens preservatives, 0.11 g BHT and 2.28 g PEG-5 Rapeseed Sterol.

The temperature was maintained at 85° C.-90° C. and mixing under medium agitation was maintained for 15-20 minutes or until homogeneous, to obtain an oil phase.

Gel phase with active in solid form

Equipment: Mixing kettle and Gaulin Mill Homogenizer

Ingredients and preparation procedure:

83.19 g Octyldodecanol were put into the Mixing Kettle under appropriate agitation.

7.31 g Bentone gelling agent, 8.77 g Allantoin and 0.73 g Propylene Carbonate were slowly added while mixing until homogeneous.

The agitation was maintained for 10-15 min.

The resulting mixture was passed through a Gaulin Mill Homogenizer to obtain a gel phase.

The gel phase was checked for appropriate the dispersion and consistency.

Additive phase

Equipment: Mixing kettle

88.03 g Ethylhexyl Methoxycinnamate were combined with 0.23 g Ascorbyl Palmitate and 11.74 g Tocopherol Acetate into the mixing kettle, mixed under appropriate agitation and heated to 60° C. to 65° C. The mixing was continued for about 10 minutes.

Preparatory phase

Equipment: Disconti Mill, Melting Kettle, Side Sweep Mixing kettle.

The preparatory phase was prepared per the following formula:

| Component | Weight in grams |
|---|---|
| Oil Phase | 43.81 |
| Diisopropyl dimer dilinoleate | 14.73 |
| Waxes | 12.63 |
| ProLipid ™ 141¹ | 0.40 |
| Gel Phase | 6.86 |
| Pigments | 4.55 |
| Silica | 1.00 |
| Mica/TiO₂ | 7.48 |
| Additive Phase | 8.54 |

¹A blend of Glyceryl Stearate, Behenyl Alcohol, Palmitic Acid, Stearic Acid, Lecithin, Lauryl Alcohol, Myristyl Alcohol, Cetyl Alcohol available from ISP, Wayne, NJ.

A portion of the Oil Phase and 14.73 g Diisopropyl Dimer Dilinoleate were combined into the Disconti Mill and heated to 65° C.

The pigments and silica were added to obtain a color phase.

The color phase was milled for 40-45 min. at 60° C. to 65° C. and checked for homogeneity.

In the melting kettle, 12.63 g of a wax mixture of Polyethylene, Ozokerite and Microcrystalline waxes and 0.4 g Prolipid 141 were melted at 95° C. to 100° C.

The color phase was discharged from the mill.

The mill was rinsed with the remaining Oil Phase for 20-30 min.

The color phase was added to the melting kettle and heated to 95° C. to 100° C.

Using a side sweep blade, the mixture in the melting kettle was mixed for 20 to 30 minutes or until homogeneous.

Mica/TiO₂ pearls (7.48 g) were added to the melting kettle and mixed with a side sweep blade for 20 to 30 minutes or until homogeneous.

The color was checked.

The temperature was lowered to 88° C. to 90° C. and 6.86 g Gel Phase were added.

Using a side sweep blade, the mixture was mixed for 20 to 30 minutes while maintaining temperature at 88° C. to 90° C.

The temperature was lowered to 85° C. to 87° C. and 8.54 g Additive Phase were added.

Using a side sweep blade, the mixture was mixed for 10 to 15 minutes or until homogeneous.

Finished product

Equipment: Side Sweep Mixing Blade

In an appropriate container, 99.75 g Preparatory Phase were mixed at 95° C. to 100° C.

The temperature was lowered to 85° C. before the fragrance (0.25 g) was added and mixed for 5 to 10 minutes.

The temperature was lowered to 80° C. to 82° C. and the batch was dropped with mixing in appropriate containers.

The lipstick composition obtained had a hardness of 135 g.

A two cell blind protomonadic product test was performed in order to compare the moisturization benefits obtained using a composition in accordance with the present invention versus a commercially available moisturizing lipstick (Maybelline® Moisture Whip Lipstick®). This test was conducted among a random sample of females aged 25-55.

All screening and placements of the first product to be tested were conducted in central location facilities. After a seven-day usage period, respondents were contacted by telephone to obtain their opinions of the first product tried and were then sent the second product to be tried. After another seven-day usage period, respondents were contacted again by telephone to obtain their product preference. Approximately 95 monadic evaluations were obtained per product in each cell among the random sample and 75 per product in each cell among Moisture Whip Lipstick® users. This yielded approximately 160 paired evaluations per cell among the general sample and 135 among Moisture Whip Lipstick® users. The study was conducted in twenty-five geographically dispersed markets.

The lipstick of the present invention was preferred over the Moisture Whip Lipstick® in regards to the perceived amount of core moisturizing benefit imparted onto the lips. Moreover, a majority of the evaluators found the lipstick of the present invention to be more comfortable on their lips and less likely to chap and/or dry out their lips as compared to the Moisture Whip Lipstick®.

What is claimed is:

1. A moisturizing cosmetic composition comprising:
(a) at least one oil gelling agent;
(b) at least one wax component;
(c) at least one polar oil;
(d) at least one active ingredient, and
(e) optionally, at least one colorant;
wherein (b) and (c) are present in a ratio by weight of from about 1:1 to about 1:5.

2. The composition of claim 1 wherein the at least one oil gelling agent is present in an amount of from about 0.1% to about 10% by weight, based on the weight of the composition.

3. The composition of claim 1 wherein the at least one wax is present in an amount of from about 0.1% to about 30% by weight, based on the weight of the composition.

4. The composition of claim 1 wherein the at least one polar oil is present in an amount of from about 0.1% to about 65% by weight, based on the weight of the composition.

5. The composition of claim 1 wherein the at least one active ingredient is present in an amount of from about 0.01% to about 10% by weight, based on the weight of the composition.

6. The composition of claim 1 wherein the at least one active ingredient is in solid form.

7. The composition of claim 1 wherein the colorant is present in an amount of from about 0.1% to about 65% by weight, based on the weight of the composition.

8. The composition of claim 1 wherein the composition has a hardness value of from about 100 g to about 200 g.

9. The composition of claim 1 wherein the composition has a hardness value of from about 130 g to about 140 g.

10. The composition of claim 1 wherein (b) and (c) are present in a ratio by weight of from about 1:2 to about 1:4.

11. The composition of claim 1 wherein (b) and (c) are present in a ratio by weight of from about 1:3 to about 1:4.

12. The composition of claim 1 wherein the at least one oil gelling agent is an organomodified clay activated with propylene carbonate.

13. The composition of claim 1 further comprising a humectant.

14. A moisturizing cosmetic composition comprising:
(a) from about 0.5 to about 1.5% by weight of at least one organomodified clay;
(b) from about 1 to about 15% by weight of a wax mixture comprising polyethylene, microcrystalline and ozokerite waxes;
(c) from about 20 to about 40% by weight of at least one polar oil chosen from plant oils, synthetic esters, fatty alcohols and mixtures thereof;
(d) from about 0.1 to about 1% by weight of allantoin; and
(e) at least one colorant;
wherein (b) and (c) are present in a ratio of from about 1:3 to about 1:4.

15. The composition of claim 14, further comprising collagen.

16. A process for moisturizing a keratinous tissue comprising contacting the tissue with a composition containing:
(a) at least one oil gelling agent;
(b) at least one wax component;
(c) at least one polar oil;
(d) at least one active ingredient; and
(e) optionally, at least one colorant;
wherein (b) and (c) are present in a ratio by weight of from 1:1 to 1:5.

17. The process of claim 16 wherein the oil gelling agent is present in an amount of from about 0.1% to about 10% by weight, based on the weight of the composition.

18. The process of claim 16 wherein the at least one wax is present in an amount from about 0.1% to about 30% by weight, based on the weight of the composition.

19. The process of claim 16 wherein the at least one polar oil is present in an amount from about 0.1% to about 65% by weight, based on the weight of the composition.

20. The process of claim 16 wherein the active ingredient is present in an amount from about 0.01% to about 10% by weight, based on the weight of the composition.

21. The process of claim 16 wherein the colorant is present in an amount from about 0.1% to about 65% by weight, based on the weight of the composition.

22. The process of claim 16 wherein the composition has a hardness value of from about 100 g to about 200 g.

23. The process of claim 16 wherein the at least one oil gelling agent is an organomodified clay activated with propylene carbonate.

24. The process of claim 16 wherein the active ingredient is in solid form.

25. A process for moisturizing a keratinous tissue comprising contacting the tissue with a composition containing:
(a) from about 0.5 to about 1.5% by weight of at least one organomodified clay;
(b) from about 1 to about 15% by weight of a wax mixture comprising polyethylene, microcrystalline and ozokerite waxes;
(c) from about 20 to about 40% by weight of at least one polar oil chosen from plant oils, synthetic esters, fatty alcohols and mixtures thereof;
(d) from about 0.1 to about 1% by weight of allantoin; and
(e) at least one colorant;
wherein (b) and (c) are present in a ratio of from about 1:3 to about 1:4.
